(19) United States
(12) Patent Application Publication
(10) Pub. No.: US 2008/0183255 A1
(43) Pub. Date: Jul. 31, 2008

(54) SIDE PORT LEAD DELIVERY SYSTEM

(75) Inventors: Mark J. Bly, Falcon Heights, MN (US); Jason A. Shiroff, Minneapolis, MN (US)

Correspondence Address: FAEGRE & BENSON, LLP;32469, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/669,050

(22) Filed: Jan. 30, 2007

(51) Int. Cl. A61N 1/05

(52) U.S. Cl. 607/116

(57) ABSTRACT

A lead delivery system for implanting a lead in a patient's internal jugular vein (IJV) through a subclavian vein. The system comprises an outer catheter having a distal portion, an intermediate portion, and a proximal portion. The outer catheter defines a lumen extending through the proximal portion to a side port located on the intermediate portion. The distal portion includes a support region for leveraging against a wall of a superior vena cava (SVC) of the patient. An inner catheter is sized to slide within the lumen and out the side port. The inner catheter includes a distal curve configured to facilitate access to the internal jugular vein. A guidewire is sized to slide within a lumen of the inner catheter. The lumen and side port are configured to direct the inner catheter towards the entrance to the IJV when the outer catheter is inserted with the support region in place against the SVC. Methods of providing access to a patient's internal jugular vein through a subclavian vein.

### CROSS-REFERENCE TO RELATED APPLICATIONS

This application is related to the following co-pending and co-owned applications: DUAL SPIRAL LEAD CONFIGURATIONS, filed on the same day and assigned Ser. No. ______; ELECTRODE CONFIGURATIONS FOR TRANSVASCULAR NERVE STIMULATION, filed on the same day and assigned Ser. No. ______; SPIRAL CONFIGURATIONS FOR INTRAVASCULAR LEAD STABILITY, filed on the same day and assigned Ser. No. ______; METHOD AND APPARATUS FOR DELIVERING A TRANSVASCULAR LEAD, filed on the same day and assigned Ser. No. ______; NEUROSTIMULATING LEAD HAVING A STENT-LIKE ANCHOR, filed on the same day and assigned Ser. No. ______; TRANSVASCULAR LEAD WITH PROXIMAL FORCE RELIEF, filed on the same day and assigned Ser. No. ______; and METHOD AND APPARATUS FOR DIRECT DELIVERY OF TRANSVASCULAR LEAD, filed on the same day and assigned Ser. No. ______, all herein incorporated by reference in their entirety.

### TECHNICAL FIELD

The present invention relates to delivery systems for medical electrical leads for nerve or muscle stimulation. The present invention more particularly relates to delivery systems and methods of providing access to a patient's internal jugular vein and adjacent to a vagus nerve.

### BACKGROUND

A significant amount of research has been directed both to the direct and indirect stimulation of nerves including the left and right vagus nerves, the sympathetic and parasympathetic nerves, the phrenic nerve, the sacral nerve, and the cavernous nerve to treat a wide variety of medical, psychiatric, and neurological disorders or conditions. More recently, stimulation of the vagus nerve has been proposed as a method for treating various heart conditions, including heart failure. Heart failure is a cardiac condition characterized by a deficiency in the ability of the heart to pump blood throughout the body and high filling pressure causing pulmonary fluid to build up in the lungs.

Typically, nerve stimulating electrodes are cuff- or impalement-type electrodes placed in direct contact with the nerve to be stimulated. These electrodes require surgical implantation and can cause irreversible nerve damage due to swelling or direct mechanical damage to the nerve. A less invasive approach is to stimulate the nerve through an adjacent vessel using an intravascular lead. A lead including one or more electrodes is inserted into a patient's vasculature and delivered to a site within a vessel adjacent a nerve to be stimulated.

Standard delivery systems exist for delivering medical electrical leads to regions in or near the heart. Problems can arise, however, when using standard delivery systems to deliver a medical electrical lead into a patient's vasculature for the purposes of nerve stimulation. One such problem occurs when a guide catheter or guidewire prolapses into the superior vena cava when attempting to access an internal jugular vein from a subclavian vein. Thus, there is a need in the art for a system for delivering a medical electrical lead into the internal jugular vein from a subclavian vein that avoids catheter or guidewire prolapse into the superior vena cava.

### SUMMARY

In one embodiment, the present invention is a lead delivery system for implanting a lead in a patient's internal jugular vein (IJV) through a subclavian vein. The system comprises an outer catheter having a distal portion, an intermediate portion, and a proximal portion. The outer catheter defines a lumen extending through the proximal portion to a side port located on the intermediate portion. The distal portion includes a support region for leveraging against a wall of a superior vena cava (SVC) of the patient. An inner catheter is sized to slide within the lumen and out the side port. The inner catheter includes a distal curve configured to facilitate access to the IJV. A guidewire is sized to slide within a lumen of the inner catheter. The lumen and side port are configured to direct the inner catheter towards an entrance to the IJV when the outer catheter is inserted with the support region in place against the SVC.

In another embodiment, the present invention is a method of providing access to a patient's internal jugular vein (IJV) through a subclavian vein. The method comprises advancing an outer catheter into the subclavian vein. The outer catheter extends from a distal portion to a proximal portion and includes a side port providing access to a lumen of the outer catheter. The side port is aligned with an entrance to the IJV. An inner catheter is advanced through the outer catheter and out the side port to a desired location. A guidewire is advanced through the inner catheter into the IJV.

In another embodiment, the present invention is a method of providing access to a patient's internal jugular vein (IJV) through a subclavian vein. The method comprises advancing an outer catheter into the subclavian vein. The outer catheter extends from a distal portion to a proximal portion and includes a side port providing access to a lumen of the outer catheter. The side port is aligned with an entrance to the IJV. A guidewire is advanced through the side port into the internal jugular vein.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a schematic view of a patient's upper torso.

FIGS. 2A-2C show side views of outer catheters for use in a delivery system according to one embodiment of the present invention.

FIG. 3 shows a side view of an inner catheter for use in a delivery system according to one embodiment of the present invention.

FIG. 4 shows a side view of a guidewire for use in a delivery system according to one embodiment of the present invention.

FIG. 5 shows a schematic view of an outer catheter, an inner catheter, and a guidewire located within a subclavian vein according to one embodiment of the present invention.

FIG. 6 shows a schematic view of an outer catheter, an inner catheter, and a guidewire after advancement of the guidewire into an internal jugular vein according to one embodiment of the present invention.

FIG. 7 shows a schematic view of outer catheter, an inner catheter, and a guidewire after advancement of the inner catheter into an internal jugular vein according to one embodiment of the present invention.

FIG. 8 is a flowchart illustrating an exemplary method of implanting a medical electrical lead into the internal jugular vein according to one embodiment of the present invention.

FIG. 9 shows a schematic view of outer catheter and a guidewire after advancement of the guidewire into an internal jugular vein according to one embodiment of the present invention.

FIG. 10 shows a schematic view of an inner catheter advanced over a guidewire into the internal jugular vein according to one embodiment of the present invention.

FIG. 11 is a flowchart illustrating an exemplary method of implanting a medical electrical lead into the internal jugular vein according to one embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

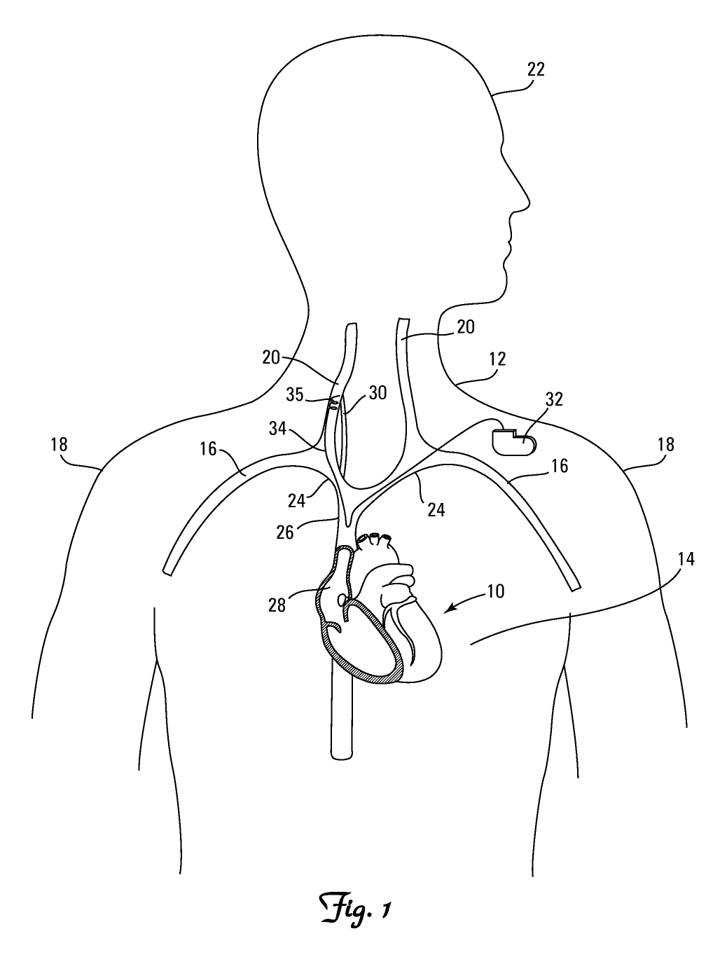

FIG. 1 shows a partial cutaway view of a patient's upper torso, including a heart 10 and the veins of the neck 12 and thorax 14. The subclavian veins 16 drain blood from the arms 18. The internal jugular veins 20 drain blood from the head 22 and join the subclavian veins 16 to form the brachiocephalic or innominate veins 24. The union of the brachiocephalic veins 24 forms the superior vena cava 26, which returns blood from the head 22, neck 12, arms 18, and thorax 14 to the right atrium 28. A vagus nerve 30 is adjacent to the right internal jugular vein 20. Another vagus nerve (not shown) is adjacent to the left internal jugular vein 20. A stimulating device 32 is located in a subcutaneous pocket near the patient's subclavian vein. The stimulating device 32 is connected to a medical electrical lead 34 extending through the patient's subclavian, brachiocephalic, and internal jugular veins. In the illustrated embodiment, the lead 34 includes a retaining structure 35 positioned in the internal jugular vein 20.

In one embodiment, the medical electrical lead 34 has the form disclosed in U.S. patent application Ser. No. ______, filed ______, ______, 2007, entitled SPIRAL CONFIGURATIONS FOR INTRAVASCULAR LEAD STABILITY, above-incorporated by reference in its entirety. In an alternative embodiment, the medical electrical lead 34 has the form of a dual spiral as disclosed in U.S. patent application Ser. No. ______, filed ______, ______, 2007, entitled DUAL SPIRAL LEAD CONFIGURATIONS, above-incorporated by reference in its entirety. In another embodiment, the medical electrical lead 34 has the form disclosed in U.S. patent application Ser. No. ______, filed ______, ______, 2007, entitled NEUROSTIMULATING LEAD HAVING A STENT-LIKE ANCHOR, above-incorporated by reference in its entirety.

In one embodiment, the stimulating device 32 includes an electrode (not shown) that provides electrical stimulation to a nerve. In one embodiment, the electrode provides electrical stimulation to a vagus nerve 30. In another embodiment, the electrode has the form disclosed in U.S. patent application Ser. No. ______, filed ______, ______, 2007, entitled ELECTRODE CONFIGURATIONS FOR TRANSVASCULAR NERVE STIMULATION, above-incorporated by reference in its entirety.

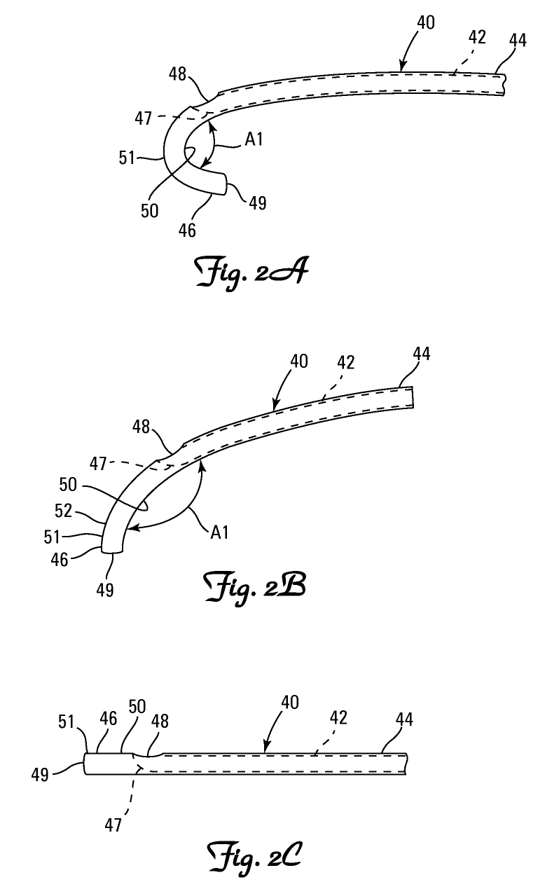

FIG. 2A shows a side view of an outer catheter 40 according to one embodiment of the present invention. The outer catheter 40 has a lumen 42, a proximal portion 44, and a distal portion 46. The lumen 42 extends from the proximal portion 44 to a side port 48, which provides access to the lumen 42. In the embodiment illustrated in FIG. 2A, the outer catheter 40 also includes a guiding feature 47 located near the side port 48. In one embodiment, the guiding feature 47 is a ramp. In other embodiments, the guiding feature 47 has any other shape adapted to guide a catheter or guidewire through the lumen 42, out the side port 48, and into the internal jugular vein 20. In the embodiment illustrated in FIG. 2A, the outer catheter 40 is generally J-shaped. The outer catheter 40 includes an intermediate portion 50 having an angle A1 extending from the side port 48 to a distal tip 49. In one embodiment, the angle A1 is between approximately 91 and approximately 180 degrees. The intermediate portion 50 includes a support region 51, which engages a wall 27 of the superior vena cava 26 when the outer catheter 40 is inserted into the subclavian vein 16 (as shown in FIG. 5). In the embodiment shown in FIG. 2A, the outside wall of the intermediate portion 50 is the support region 51. In another embodiment, the support region 51 engages a wall of the brachiocephalic vein 24.

FIG. 2B illustrates an outer catheter 40 according to another embodiment of the present invention. The outer catheter 40 includes an intermediate portion 50 having an angle A1 extending from the side port 48 to the distal tip 49. In the embodiment illustrated in FIG. 2B, the angle A1 is between approximately 1 and approximately 90 degrees. The outer catheter 40 includes a drop-down portion extending from the intermediate portion 50 to the distal tip 49. The drop-down portion 52 is sized to extend into the superior vena cava 26 when the outer catheter 40 is advanced into the brachiocephalic vein 24 and includes a support region 51 that engages a superior vena cava wall 27 when the outer catheter 40 is located into the subclavian vein 16. In one embodiment, the drop-down portion 52 has a length between approximately 1 and approximately 8 centimeters. FIG. 2C illustrates an outer catheter 40 according to yet another embodiment of the present invention. The outer catheter 40 includes an intermediate portion 50 that is substantially straight. In the embodiment shown in FIG. 2C, the support region 51 for engaging the superior vena cava wall 27 is the distal portion 46. In another embodiment, the outer catheter 40 includes a series of curves. In yet another embodiment, the outer catheter 40 includes an S-shaped series of curves.

In the embodiments illustrated in FIGS. 2A-2C, the outer catheter 40 has a solid cross-section from the guiding feature 47 to the distal tip 49. In other embodiments, the region between the guiding feature 47 and the distal tip 49 includes a lumen (not shown). In one embodiment, the side port 48 has a diameter substantially equivalent to the diameter of the lumen 42. In one embodiment, the outer catheter 40 has a length between approximately 20 and approximately 40 centimeters. In one embodiment, the outer catheter 40 has an outer diameter between approximately 6 and approximately 14 French, and an inner diameter slightly less than the outer diameter.

In another embodiment, the angle A1, length of the outer catheter, location of the side port 48, dimensions of the intermediate portion 40, and other characteristics of the outer catheter 40 are selected based on the patient's anatomy such that the support region 51 engages the superior vena cava wall 27 to align the side port 48 with the entrance of a brachiocephalic vein 24 when the outer catheter 40 is located in the subclavian vein 16. In another embodiment, the characteristics of the outer catheter 40 are selected to align the side port 48 with the entrance of a brachiocephalic vein 24 when inserted in a subclavian vein 16 without engaging a support region 51 with the wall 27 of the superior vena cava 26. This alignment prevents prolapse of a guidewire 70 or inner catheter 60 into the superior vena cava 26. The alignment of the side port 48 with the entrance of the brachiocephalic vein 24 also aligns the side port 48 with the entrance to the internal jugular vein 20. In yet another embodiment, where the implantation is a “same side” implantation, the location of the side port 48 is selected so that the side port 48 is directly aligned with the entrance to the internal jugular vein 20 and the inner catheter directly accesses the internal jugular vein 20 from the subclavian vein 16.

FIG. 3 illustrates an inner catheter 60 according to one embodiment of the present invention. The inner catheter 60 includes a lumen 62, which extends from a proximal portion 64 to a distal portion 66. In one embodiment, the inner catheter 60 has a length between approximately 30 and approximately 80 centimeters. The inner catheter 60 has an outer diameter that allows it to slide within the lumen 42 and out the side port 48 of the outer catheter 40. In one embodiment, the inner catheter has an outer diameter between approximately 4 and approximately 12 French, and an inner diameter slightly less than the outer diameter. In the embodiment shown in FIG. 3, the inner catheter 60 has a curve 68 near its distal portion 66. In one embodiment, the curve 68 has an angle A2 between approximately 40 and approximately 120 degrees, and is located between approximately 1 and approximately 2 centimeters from a distal tip 69 of the inner catheter 60. In other embodiments, the inner catheter 60 does not have a curve.

The inner and outer catheters 40, 60 can be made of a polytetrafluoroethylene (PTFE) or fluoronated ethylene propylene (FEP) inner lining, a 304 V stainless steel braiding, and an outer jacket of Pebax and/or Nylon. Tungsten wire can optionally be added to the stainless steel braiding to improve radiopacity of the catheter. In other embodiments, the inner and outer catheters 60, 40 are comprised of any other material known in the art.

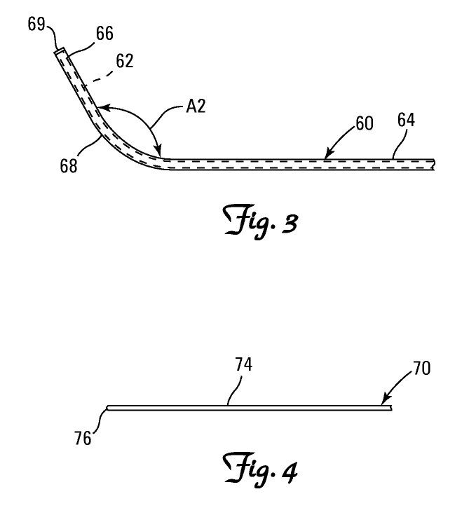

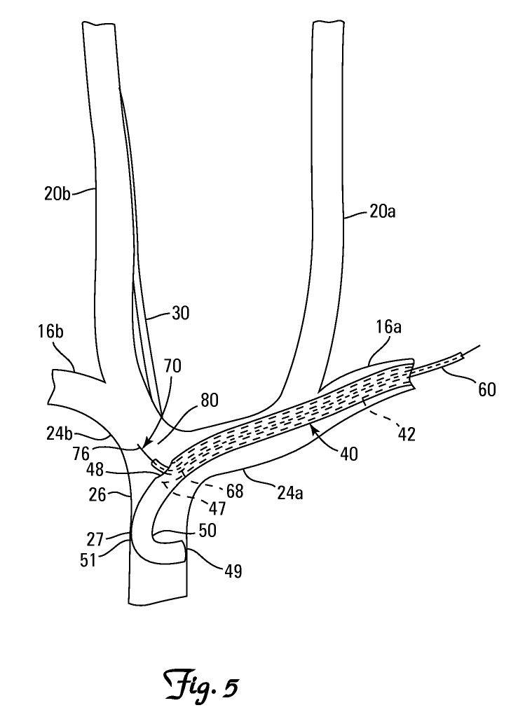

FIG. 4 depicts a guidewire 70 according to one embodiment of the present invention. In the illustrated embodiment, the guidewire 70 includes a body 74 and a distal tip 76. The guidewire 70 allows a clinician to introduce and position a catheter or a medical electrical lead 34 in a patient. In one embodiment, the guidewire 70 has a core (not shown) and includes a coating, such as a hydrophilic coating. The guidewire 70 has an outer diameter that allows it to slide within the lumen 62 of the inner catheter 60. In one embodiment, the outer diameter is between approximately 0.012 and approximately 0.040 inch. In one embodiment, the length of the guidewire 70 is between approximately 100 and approximately 250 centimeters. The guidewire 70 is shown as substantially straight in FIG. 4, but can have a J shape in other embodiments. In one embodiment, the distal tip 76 is an atraumatic tip.

FIG. 5 is a schematic view showing the outer catheter 40, inner catheter 60, and guidewire 70 located in the subclavian vein 16. In the illustrated embodiment, the outer catheter 40 is positioned in the left subclavian vein 16a to facilitate implantation of a medical electrical lead 34 in the opposite, or right, internal jugular vein 20b. Although the method of implantation is described with respect to the left subclavian vein 16a and right internal jugular vein 20b, in other embodiments, the method is used to implant a medical electrical lead 34 in the left internal jugular vein 20a from the right subclavian vein 16b. In other embodiments, the method of implantation is a “same side” implantation from the right subclavian vein 16b into the right internal jugular vein 20b, or the left subclavian vein 16a into the left internal jugular vein 20a. In one embodiment, the outer catheter 40 is inserted into the left subclavian vein 16a using a percutaneous venipuncture. In an alternative embodiment, the outer catheter 40 could be inserted using a surgical cut-down to the subclavian vein 16 from a subcutaneous pocket (not shown) created for stimulating device 32, or in any other manner as is known in the art.

In the embodiment shown in FIG. 5, the outer catheter 40 is advanced through the left subclavian vein 16a so that the side port 48 is aligned with the entrance 80 to the right brachiocephalic vein 24b from the superior vena cava 26. Although the outer catheter 40 is shown as generally J-shaped in FIG. 5, in other embodiments, other outer catheter 40 configurations are used. The side port 48 prevents the inner catheter 60 and the guidewire 70 from prolapsing into the superior vena cava 26 during implantation. The intermediate portion 50 and the engagement of the outer catheter support region 51 with the superior vena cava wall 27 facilitate this alignment. The support region 51 is leveraged against the superior vena cava wall 27 and closes any gap between the support region 51 and the superior vena cava wall 27. In the illustrated embodiment, the lumen 42, the side port 48, and the guiding feature 47 guide the inner catheter 60 and the guidewire 70 out of the side port 48 and into the internal jugular vein 20b. The inner catheter curve 68 directs the guidewire 70 into the internal jugular vein 20b.

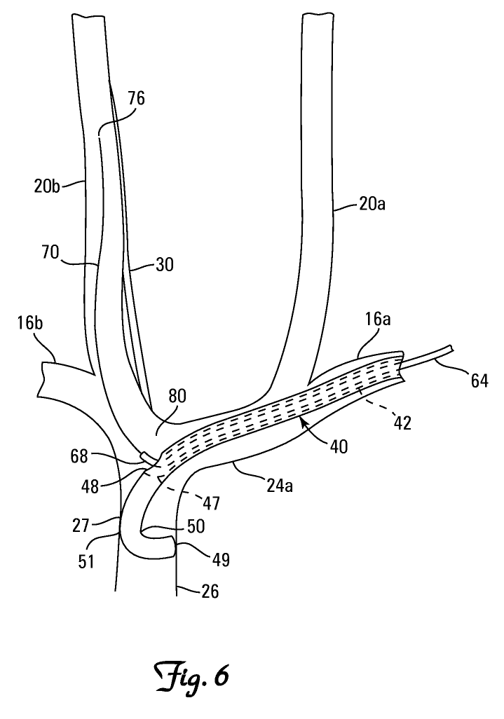

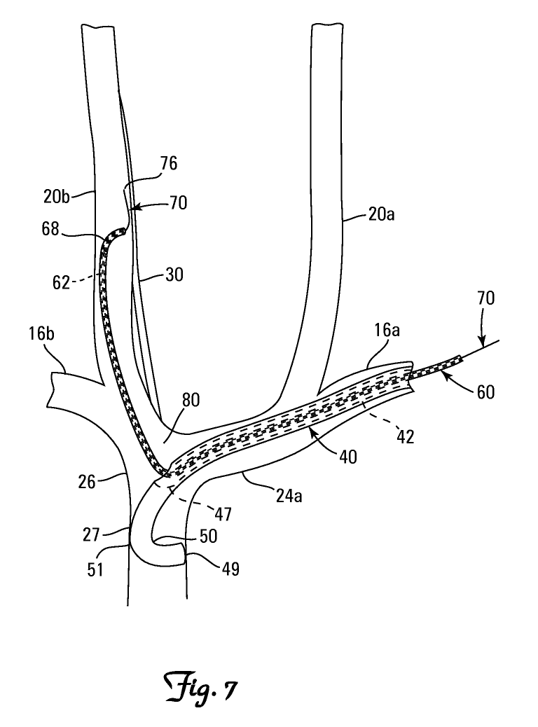

As shown in FIG. 5, the outer catheter 40 has a solid cross-section from the guiding feature 47 to the distal tip 49, thereby preventing the guidewire 70 from passing out the distal tip 49 into the superior vena cava 26. The distal tip 49 may be soft or atraumatic to avoid damaging the blood vessels during the implantation process. In one embodiment, the region between the distal tip 49 and the side port 48 is soft or atraumatic. In one embodiment, the distal tip 49 may be radiopaque to facilitate alignment of the side port 48 with the entrance 80. FIG. 6 is a schematic view showing the guidewire 70 after advancement through the lumen 62 of the inner catheter 60 to a suitable location in the right internal jugular vein 20b. In alterative embodiments, the guidewire 70 is advanced to any desired location in the vasculature. FIG. 7 is a schematic view of the inner catheter 60 when advanced over the guidewire 70 into the right internal jugular vein 20b.

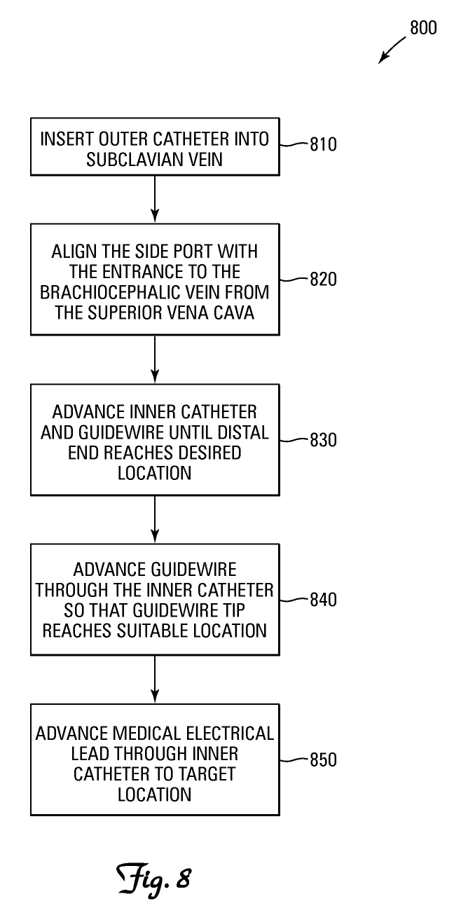

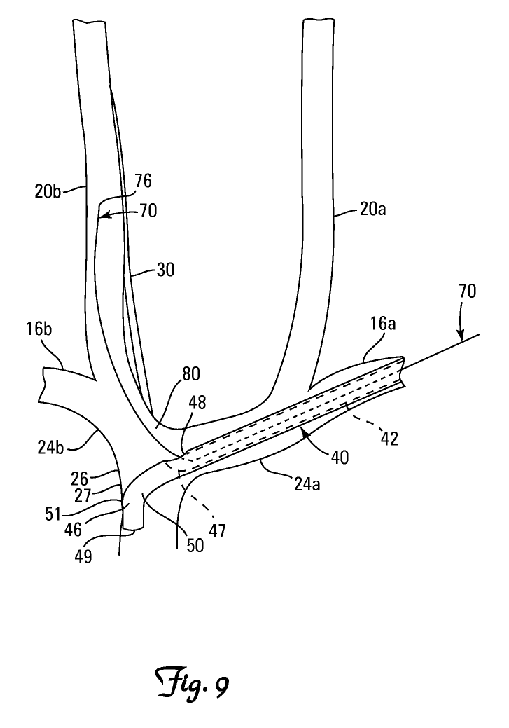

FIG. 8 is a flowchart illustrating an exemplary method 800 of implanting a medical electrical lead 34 in an internal jugular vein 20 from a subclavian vein 16 using an outer catheter 40, an inner catheter 60, and a guidewire 70. The outer catheter 40 is inserted into the subclavian vein 16 (block 810). The side port 48 is aligned with the entrance 80 to the brachiocephalic vein 24 from the superior vena cava 26 (block 820). The outer catheter 40 can be secured in the subclavian vein 16 using a suture or in any other manner as is known in the art. The inner catheter 60 is advanced through the outer catheter 40 to a desired location (block 830). In one embodiment, the desired location is in the brachiocephalic vein 24. In another embodiment, the desired location is in the internal jugular vein 20. In one embodiment, a guiding feature 47 is used to guide the inner catheter 60 out of the side port 48 and to the desired location. In one embodiment, the inner catheter 60 is advanced over the guidewire 70 and through the lumen 42 to the desired location. In another embodiment, the inner catheter 60 is advanced to the desired location and the guidewire 70 then advanced through the inner catheter 60 to the desired location. Next, the guidewire 70 is advanced through the inner catheter 60 so that the guidewire tip 76 reaches a suitable location in the internal jugular vein 20 (block 840). A medical electrical lead 34 is then advanced through the inner catheter 60 to a target location in the internal jugular vein 20 (block 850).

In one embodiment, the medical electrical lead 34 includes a lumen and is advanced over the guidewire 70. In one embodiment, after the guidewire 70 is advanced through the inner catheter 60 to the suitable location in the internal jugular vein 20, the inner catheter 60 is advanced over the guidewire 70 from a desired location in the brachiocephalic vein 24 into the internal jugular vein 20. In another embodiment, the outer catheter 40 is removed after the guidewire 70 reaches a suitable location and the medical electrical lead 34 is advanced through the inner catheter 60 to a target location. In yet another embodiment, the outer catheter 40 is removed and a third catheter (not shown) advanced over the inner catheter 60. The inner catheter 60 is then removed and the medical electrical lead 34 is advanced through the third catheter to the target location. In other embodiments, venograms are taken through either the inner catheter 60 or the outer catheter 40. In one embodiment, a contrast fluid or contrast fluid/saline mixture is injected into the proximal portion 44 of the outer catheter 40 and exits through the side port 48, thereby allowing a clinician to visualize the anatomy using a venogram. In another embodiment, the implantation is a “same side” implantation and the outer catheter 40 is inserted into the subclavian vein 16 so that the side port 48 is directly aligned with the entrance to the internal jugular vein 20 and the inner catheter directly accesses the internal jugular vein 20 from the subclavian vein 16.

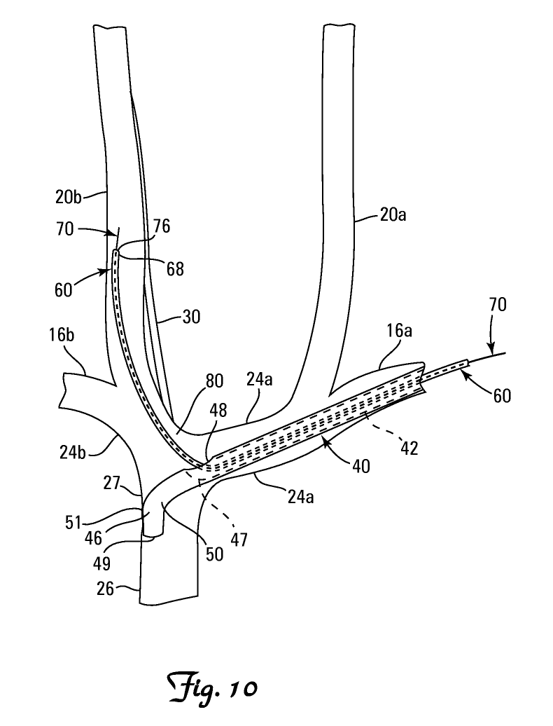

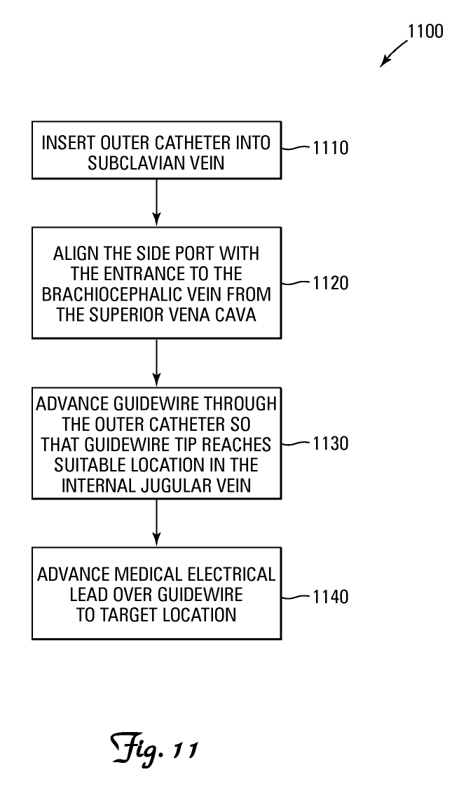

FIG. 9 illustrates an outer catheter 40 having a substantially straight intermediate portion 50 inserted into the left subclavian vein 16a and advanced so that the side port 48 is aligned with the entrance 80 to the right brachiocephalic vein 24b from the superior vena cava 26. The outer catheter 40 shown in FIG. 9 has an intermediate portion 50 that is initially substantially straight. As shown in FIG. 9, when the distal tip 49 reaches the superior vena cava wall 27, the distal portion 46 bends and the support region 51 engages the superior vena cava wall 27, thereby aligning the side port 48 with the entrance 80. The guidewire 70 is inserted through the lumen 42 of the outer catheter 40 and out the side port 48 so that the guidewire distal tip 76 reaches a suitable location in the right internal jugular vein 20b. As shown in FIG. 10, the inner catheter 60 is advanced over the guidewire 70 through the lumen, and out the side port 48 to a desired location in the right internal jugular vein 20b. The medical electrical lead 34 can then be advanced through the inner catheter 60 to a target location in the right internal jugular vein 20b using a variety of techniques.

FIG. 11 is a flowchart illustrating an exemplary method 1100 for implanting a medical electrical lead 34 in an internal jugular vein 20 from a subclavian vein 16 using an outer catheter 40 and a guidewire 70. The outer catheter 40 is inserted into the subclavian vein 16 (block 1110). The side port 48 is aligned with the entrance 80 to the brachiocephalic vein 24 from the superior vena cava 26 (block 1120). The guidewire 70 is advanced through the outer catheter 40 so that the tip 76 reaches a suitable location in the internal jugular vein 20 (block 1130). A medical electrical lead 34 is advanced over the guidewire 70 to a target location in the internal jugular vein 20 (block 1140).

In another embodiment, after the guidewire 70 is advanced into the internal jugular vein 20, an inner catheter 60 is advanced over the guidewire 70 to a desired location in the internal jugular vein 20. In yet another alternative embodiment, a guiding feature 47 is used to guide the inner catheter 60 or the guidewire 70 out of the side port 48. In one embodiment, the inner catheter 60 does not have a curve and the brachiocephalic vein 24 accommodates the configuration of the inner catheter 60 as needed. The medical electrical lead 34 is then implanted through the inner catheter 60. In yet another alternative embodiment, the outer catheter 40 is removed before the medical electrical lead 34 is advanced to the target location. In another embodiment, the implantation is a “same side” implantation and the outer catheter 40 is inserted into the subclavian vein 16 so that so that the side port 48 is directly aligned with the entrance to the internal jugular vein 20 and the inner catheter directly accesses the internal jugular vein 20 from the subclavian vein 16. In other embodiments, venograms are taken through either the inner catheter 60 or the outer catheter 40. In one embodiment, a contrast fluid or contrast fluid/saline mixture is injected into the proximal portion 44 of the outer catheter 40 and exits through the side port 48, thereby allowing a clinician to visualize the anatomy using a venogram.

The invention facilitates orientation of the lead 34 and an electrode (not shown) within the internal jugular vein 20. In one embodiment, when the guidewire 70 is inserted into the lead 34, the guidewire 70 reduces the force exerted by the retaining structure 35 on a surface external to the retaining structure, for example, the outer catheter 40, the inner catheter 60, or the internal jugular vein 20, thereby facilitating advancement and orientation of the lead 34. In another embodiment, when a portion of the retaining structure 35 remains in the outer catheter 40 or the inner catheter 60, the outer or inner catheter 40, 60 is used to rotate the lead 34 and position the electrode proximal to a vagus nerve 30.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A lead delivery system for implanting a lead in a patient's internal jugular vein (IJV) through a subclavian vein, the system comprising:
an outer catheter having a distal portion, an intermediate portion, and a proximal portion, the outer catheter defining a lumen extending through the proximal portion to a side port located on the intermediate portion, the distal portion including a support region for leveraging against a wall of a superior vena cava (SVC) of the patient;
an inner catheter sized to slide within the lumen and out the side port, the inner catheter including a distal curve configured to facilitate access to the IJV; and
a guidewire sized to slide within a lumen of the inner catheter;
wherein the lumen and the side port are configured to direct the inner catheter towards an entrance to the IJV when the outer catheter is inserted with the support region in place against the SVC.

2. The lead delivery system of claim 1 wherein the outer catheter includes a guiding feature adapted to guide the inner catheter out of the side port.

3. The lead delivery system of claim 2 wherein the guiding feature comprises a ramp.

4. The lead delivery system of claim 1 wherein the intermediate portion has an angle between approximately 91 and 180 degrees and an outside wall of the intermediate portion comprises the support region.

5. The lead delivery system of claim 1 wherein the intermediate portion has an angle between approximately 1 and approximately 90 degrees and includes a drop down portion, and the support region is located on the drop down portion.

6. The lead delivery system of claim 1 wherein the intermediate portion is substantially straight and the support region is the distal portion of the outer catheter.

7. The lead delivery system of claim 1 wherein the inner catheter distal curve has an angle between approximately 40 and approximately 120 degrees.

8. The lead delivery system of claim 1 wherein a portion of the outer catheter extending from the side port to a distal tip of the outer catheter is solid.

9. A method of providing access to a patient's internal jugular vein (IJV) through a subclavian vein, the method comprising:
advancing an outer catheter into the subclavian vein, the outer catheter extending from a distal portion to a proximal portion and including a side port providing access to a lumen of the outer catheter;
aligning the side port with an entrance to the IJV;
advancing an inner catheter through the outer catheter and out the side port to a desired location; and
advancing a guidewire through the inner catheter into the IJV.

10. The method of claim 9 wherein the method further comprises leveraging a support region located on the distal portion of the outer catheter against a wall of the SVC.

11. The method of claim 9 wherein the method further comprises leveraging a support region located on the distal portion of the outer catheter against a wall of the brachiocephalic vein.

12. The method of claim 9 wherein the method further comprises directly aligning the side port with the entrance to the IJV so that the inner catheter directly accesses the IJV from the subclavian vein.

13. The method of claim 9 wherein the method further comprises aligning the side port with an entrance to a brachiocephalic vein from a superior vena cava to align the side port with the entrance to the IJV.

14. The method of claim 9 wherein advancing the inner catheter comprises advancing the inner catheter to a desired location in the internal jugular vein.

15. The method of claim 9 wherein advancing the inner catheter comprises advancing the inner catheter to a desired location in the brachiocephalic vein and the method further comprises advancing the inner catheter over the guidewire to a desired location in the internal jugular vein.

16. The method of claim 9 further comprising advancing a medical electrical lead through a lumen of the inner catheter to a target location in the internal jugular vein.

17. The method of claim 16 further comprising advancing the medical electrical lead over the guidewire to the target location.

18. The method of claim 16 wherein the outer catheter is removed before the medical electrical lead is advanced through the lumen of the inner catheter.

19-20. (canceled)

21. A method of providing access to a patient's internal jugular vein through a subclavian vein, the method comprising:
advancing an outer catheter into the subclavian vein, the outer catheter extending from a distal portion to a proximal portion and including a side port providing access to a lumen of the outer catheter;
aligning the side port with an entrance to the IJV; and
advancing a guidewire through the side port into the IJV.

22. The method of claim 21 wherein the method further comprises leveraging a support region located on the distal portion of the outer catheter against a wall of the SVC.

23. The method of claim 21 wherein the method further comprises leveraging a support region located on the distal portion of the outer catheter against a wall of the brachiocephalic vein.

24. The method of claim 21 wherein the method further comprises aligning the side port with the entrance to the IJV so that the inner catheter directly accesses the IJV from the subclavian vein.

25. The method of claim 21 wherein the method further comprises aligning the side port with an entrance to a brachiocephalic vein from a superior vena cava to align the side port with the entrance to the IJV.

26. The method of claim 21 further comprising advancing the medical electrical lead over the guidewire to a target location in the internal jugular vein.

27. The method of claim 21 further comprising:
advancing an inner catheter over the guidewire; and
advancing a medical electrical lead through the inner catheter to a target location in the internal jugular vein.

28. (canceled)
